(19) United States
(12) Patent Application Publication
(10) Pub. No.: US 2019/0261999 A1
(43) Pub. Date: Aug. 29, 2019

(54) DEVICES FOR DELIVERY OF TREATMENT AGENTS AND RELATED METHODS

(71) Applicant: Boston Scientific Scimed, Inc., Maple Grove, MN (US)

(72) Inventors: John MURPHY, Cork (IE); Daniel Sexton, Douglas (IE); Deirdre Barrett, Ovens (IE); Jillian Lee, Ballincollig (IE); Kevin Carbery, Fermoy (IE); Samuel Raybin, Marlborough, MA (US); Matthew B. Hollyer, Watertown, MA (US); Conor O'Sullivan, Wilton (IE)

(73) Assignee: Boston Scientific Scimed, Inc., Maple Grove, MN (US)

(21) Appl. No.: 16/284,168

(22) Filed: Feb. 25, 2019

Related U.S. Application Data
(60) Provisional application No. 62/634,953, filed on Feb. 26, 2018.

(51) Int. Cl. A61B 17/12; A61L 31/04

(52) U.S. Cl. CPC A61B 17/1215; A61B 2017/00526; A61L 31/042; A61B 17/12145

(57) ABSTRACT

A medical device may include a helical coil having a plurality of turns. The plurality of turns may define a lumen. The device may also include a composition. The composition may include gellan gum, at least one salt, water, and a treatment agent. The treatment agent may be disposed in the lumen.

### CROSS-REFERENCE TO RELATED APPLICATION(S)

This application claims the benefit of priority from U.S. Provisional Application No. 62/634,953, filed on Feb. 26, 2018, which is incorporated by reference herein in its entirety.

### TECHNICAL FIELD

Various aspects of the present disclosure relate generally to devices for delivery of treatment agents and related methods. More specifically, the present disclosure relates to implantable devices including carriers which carry treatment agents, and methods related to such devices.

### BACKGROUND

For treatment of various medical conditions, it may be desirable to implant a device in a subject's body. In one form of treatment, a device may be implanted to occlude a body lumen. For example, blood vessels may be occluded to limit blood flow within an aneurysm, arteriovenous malformation, or arteriovenous fistula. Occlusion may also be helpful for, for example, controlling bleeding (e.g., from a hemorrhage) or blocking blood flow to a tumor. Implantable devices may be used to assist in producing such occlusion. For example, occlusion coils may be used. Such coils may, for example, encourage clotting.

It may also be desirable to deliver a treatment agent at a target location. However, delivery of treatment agents systemically may require larger dosages and/or may result in circulation of the treatment agent. Certain treatment agents may carry unpleasant side effects, making widespread circulation undesirable. Therefore, mechanisms to effectively deliver treatments to target locations without system-wide circulation may improve outcomes. Moreover, targeting delivery of treatment agents by containing them in a local area, and/or by controlling their rate of administration, also may be useful.

### SUMMARY

Aspects of the present disclosure relate to, among other things, devices for delivery of treatment agents and methods thereof. Each of the aspects disclosed herein may include one or more of the features described in connection with any of the other disclosed aspects.

In one example, medical device may include a helical coil having a plurality of turns. The plurality of turns may define a lumen. The device may also include a composition. The composition may include gellan gum, at least one salt, water, and a treatment agent. The treatment agent may be disposed in the lumen.

Any example of medical devices described herein may additionally or alternatively include one or more of the features below. The treatment agent may be a thrombogenic agent or a therapeutic agent. The helical coil may include at least a first region and at least a second region. A distance between turns in the first region may differ from a distance between turns in the second region. The composition may be disposed in the first region and the second region. The medical device may be operative to elute at least a portion of the composition disposed in the first region at a first rate when the device is delivered to a location within a body. The medical device may be operative to elute at least a portion of the composition disposed in the second region at a second rate when the device is delivered to the location within the body. The first rate differs from the second rate. The distance between turns in the first region may be at least approximately 1.5 times as large as the distance between turns in the second region. The distance between turns in the first region may be at least approximately 2.0 times as large as the distance between turns in the second region. The helical coil may include a third region. A distance between turns in the third region may differ from the distance between turns in the first region and the distance between turns in the second region. The composition may be a first composition and the treatment agent may be a first treatment agent. The medical device may further include a second composition disposed in the lumen. The first composition may differ from the second composition. The first composition may have a first elution rate and the second composition may have a second elution rate, and wherein the first elution rate differs from the second elution rate. The second composition may include a second treatment agent. The first treatment agent may differ from the second treatment agent. The composition may include 0.01% to 2.0% gellan gum by weight with respect to a total weight of the composition.

In another example, a medical device may include an outer region defining a lumen. The outer region may terminate at a first free end and at a second free end. The first and second free ends may be on opposite ends of the outer region. A composition may be disposed in the lumen. The composition may include 0.01% to 2.0% gellan gum by weight with respect to a total weight of the composition, at least one salt, water, and a treatment agent. The device may be operative to elute the treatment agent through one or more gaps in the outer region.

Any example of medical devices described herein may additionally or alternatively include one or more of the features below. The outer region may include a first gap and a second gap. The first gap may be larger than the second gap. At least one of the first free end and the second free end may include a tip.

In another example, a method of preparing a medical device may include introducing a composition into the medical device. The medical device may include a helical coil having a plurality of turns. The turns of the coil may define a lumen for receiving the composition. The composition may include gellan gum, at least one saltwater, and a treatment agent.

Any method described herein may include one or more of the features or steps described below. The introducing step may occur immediately prior to a procedure for implanting the medical device in a body. The helical coil may include at least a first region and at least a second region. A distance between turns in the first region may differ from a distance between turns in the second region. The introducing step may include infusing the composition in the first region and the second region. The medical device may be operative to elute at least a portion of the composition disposed in the first region at a first rate when the device is delivered to a location within a body. The medical device may be operative to elute at least a portion of the composition disposed in the second region at a second rate when the device is delivered to the location within the body. The first rate may differ from the second rate. The composition may be a first composition and the treatment agent may a first treatment agent. The method may further include introducing a second composition into the lumen of the medical device. The first composition may differ from the second composition. The first composition may have a first elution rate and the second composition may have a second elution rate. The first elution rate may differ from the second elution rate.

### BRIEF DESCRIPTION OF THE DRAWINGS

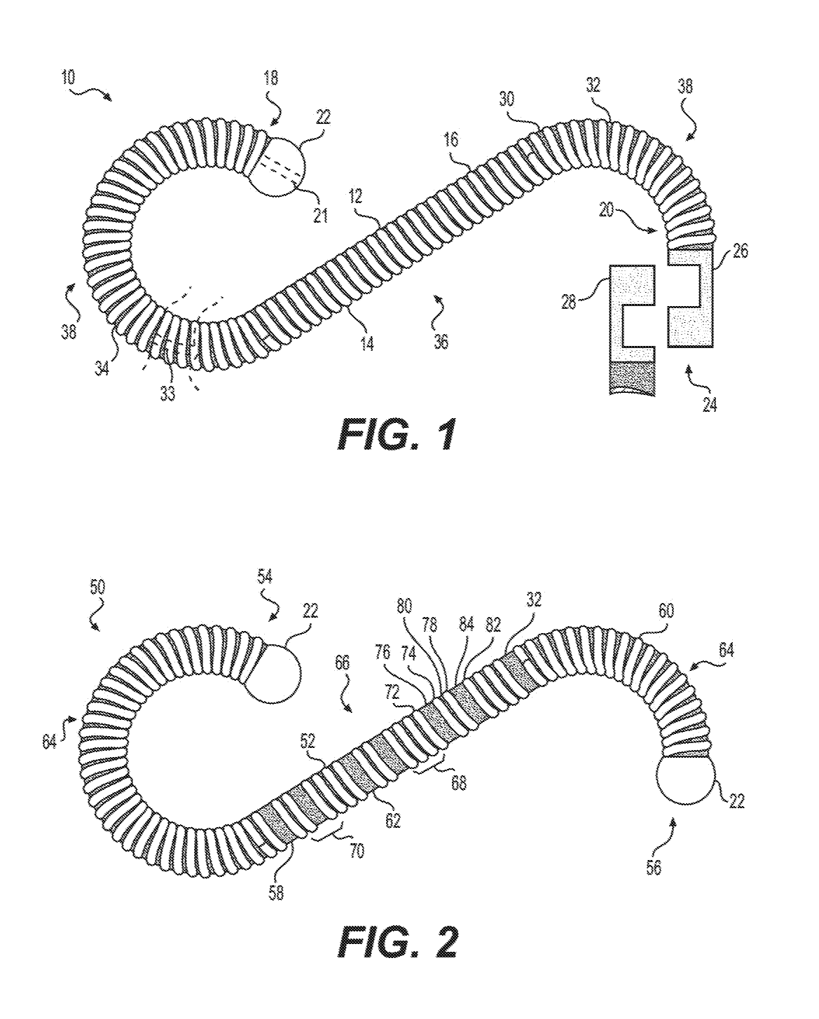

The accompanying drawings, which are incorporated in and constitute a part of this specification, illustrate aspects of the present disclosure and together with the description, serve to explain the principles of the disclosure. The drawings, FIGS. 1-10, show features of several exemplary devices for delivering treatment agents, according to aspects of the present disclosure.

### DETAILED DESCRIPTION

The present disclosure is drawn generally to devices for delivery of treatment agents and related methods, and more specifically to implantable devices including carriers for carrying treatment agents and methods related to such devices.

Reference now will be made in detail to aspects of the present disclosure, examples of which are illustrated in the accompanying drawings. Wherever possible, the same reference numbers will be used throughout the drawings to refer to the same or like parts. Though the following description refers to “occlusion,” the principles/aspects described herein may be applicable in other contexts not limited to “occlusion.” It may be understood that both the foregoing general description and the following detailed description are exemplary and explanatory only and are not restrictive of the features claimed. Further, as used herein, the terms “comprises,” “comprising,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” The terms “substantially,” “approximately” and “about” refer to a variation of plus or minus ten percent with respect to a stated value. The present embodiments disclosed herein may be used independently or in combination with one or more other disclosed embodiments.

FIG. 1 depicts an implantable device 10 for delivery of one or more treatment agents to a target location in a body of a subject. Device 10 may be used to occlude a body lumen. The target location may be in a subject's peripheral vasculature, cardio vasculature, neurovascular system, and/or gastrointestinal system, among other places. Device 10 may be implanted in a subject during a stand-alone procedure, or in a combined procedure for use in conjunction with one or more other devices. The features of device 10 discussed herein may also apply to the other devices discussed below.

Device 10 may include a coil 12. Coil 12 may include a linear element 14. Linear element 14 may have any suitable cross-section, such as a circular or polygonal cross-section. Linear element 14 may be, for example, a wire. Linear element 14 may be shaped into coil 12, which may contain a plurality of windings 16. Coil 12 may be made of any suitable material. For example, coil 12 may be made of metals, super-elastic alloys (e.g. nitinol), and/or polymers. Coil 12 may include a combination of the aforementioned materials, for example, a wire wrapped with a material such as a polymer. The width or diameter of the wire or other material making up coil 12 may be any suitable width or diameter, for example 0.005-0.050 inches. It is also contemplated that different regions of coil 12 may be made of different materials, and/or may have wire or other material with different widths or diameters. Coil 12 may also include fibrous material (not shown) attached thereto to facilitate occlusion.

Device 10 may have a first end portion 18 and a second end portion 20. First and second end portions 18 and 20 may be at opposite ends of device 10. Device 10 may have one or more tips 22 at one or more of first end portion 18 and/or second end portion 20. Tip 22 may be an atraumatic tip, a cap, a welding, a welding deposit, a seed marker, and/or any suitable structure. Alternatively, coil 12 may have an open end (not shown) without tip 22.

In one example, device 10 may include at least a portion of a delivery device 24 at one or both of first end portion 18 and second end portion 20. Delivery device 24 may include, for example, a first section 26 which may be fixedly attached to coil 12 or another component of device 10, and a second section 28 which may engage with first section 26 while device 10 is guided toward and/or placed at a target location, second section 28 being configured to be manipulated by a user to control device 10. After device 10 is placed, first section 26 may disengage from second section 28 to leave device 10 at the target location, and to allow second section 28 to be removed. A length of device 10 may be described as the dimension of device 10 measured in a direction extending from first end portion 18 toward second end portion 20. For example, the length of device 10 may be measured from an endmost portion of tip 22 to an endmost portion of first section 26.

Coil 12 may be helically wound. To form coil 12, linear element 14 may be, for example, wrapped around a shaping device such as a mandrel or stylet. Coil 12 may be close-coiled or open-coiled. If coil 12 is close-coiled, adjacent windings 16 may abut one another. If coil 12 is open-coiled, there may be space between adjacent windings or turns 16. The amount of space (if any) between windings 16 may be uniform along the length of coil 12 or may be variable, as discussed in further detail below.

Windings 16 of coil 12 may define a lumen 30 running lengthwise through coil 12. Lumen 30 may have a central axis along the length of device 10. Lumen 30 may have a cross-section of any suitable shape. For example, lumen 30 may have a circular or polygonal cross-sectional shape. The diameter or width of lumen 30 may be any suitable amount, such as 0.010-0.023 inches. Lumen 30 may alternatively have any other diameter or width. The width or diameter of lumen 30, and/or the cross-sectional shape of lumen 30, may be uniform along a central axis of lumen 30 or may be variable.

A carrier 32 may be disposed in lumen 30. At least a portion of carrier 32 may be completely or partially biodegradable or absorbable. At least a portion of carrier 32 may include or be made of a bioactive material or agent. For example, carrier 32 may include a composition including at least one gelling agent, at least one salt, and water. The at least one gelling agent may be natural (including natural gums such as vegetable gums and/or microbial gums) or synthetic in origin, and may be anionic, cationic, or neutral. Non-limiting examples of gelling agents suitable for the compositions herein include polysaccharides such as gellan gum, xanthan gum, gum arabic, guar gum, locust bean gum, alginate, and carrageenans. The concentration of gelling agent(s) may range from about 0.01% to about 2.0% by weight with respect to the total weight of the composition, such as from about 0.02% to about 1.5%, from about 0.05% to about 0.5%, from about 0.10% to about 1.0%, from about 0.10% to about 0.30%, or from about 0.02% to about 0.25% by weight with respect to the total weight of the composition, e.g., about 0.10%, about 0.15%, or about 0.20% by weight with respect to the total weight of the composition. In at least one embodiment, the total concentration of gelling agent(s) in the composition ranges from about 0.05% to about 0.5% by weight with respect to the total weight of the composition.

The at least one gelling agent may be combined with one or more biocompatible, e.g., physiologically compatible, salts. Non-limiting examples of salts suitable for the compositions herein include salts comprising sodium, calcium, magnesium, and/or potassium cations. The salts may include, for example, chloride salts, phosphate salts, and/or sulfate salts, such as, e.g., sodium chloride (NaCl), potassium chloride (KCl), calcium chloride (CaCl₂), sodium dihydrogen phosphate (NaH₂PO₄), potassium hydrogen phosphate (K₂HPO₄), magnesium sulfate (MgSO₄), sodium gluconate (C₆H₁₁NaO₇), sodium acetate trihydrate (C₂H₉NaO₅·3H₂O), and magnesium chloride (MgCl₂). For example, the at least one gelling agent may comprise an anionic polysaccharide, and the salt(s) may provide a source of monovalent or divalent cations compatible with the polysaccharide.

In some embodiments, the composition may comprise a physiologically compatible saline solution, such as, e.g., a sodium chloride solution. For example, the composition may comprise a 0.9% wt. sodium chloride solution, e.g., providing sodium cations to assist in formation of the three-dimensional solid gel network. In some embodiments, the composition may be isotonic. For example, the saline solution may have an appropriate concentration of monovalent and/or divalent cations such that the composition is isotonic with tissue fluids and/or blood. Other physiologically-compatible solutions comprising suitable ionic concentrations may be used to provide for isotonicity.

The concentration of salt of the composition may range from about 0.1% to about 2.0% by weight with respect to the total weight of the composition, such as from about 0.25% to about 1.0%, from about 0.5% to about 1.5%, or from about 0.5% to about 1.0% by weight with respect to the total weight of the composition, e.g., about 0.25%, about 0.5%, about 0.75%, or about 1.0% by weight with respect to the total weight of the composition. Further, for example, the composition may comprise a salt solution having an osmolality ranging from about 240 mOsmol/kg to about 340 mOsmol/kg (e.g., 290 mOsmol/kg±50 mOsmol/kg), such as from about 250 mOsmol/kg to about 320 mOsmol/kg, or from about 280 mOsmol/kg to about 300 mOsmol/kg. The solution may be physiologically compatible, e.g., having electrolyte levels, osmolality, and pH suitable for injection into a patient. The pH of the solution may be adjusted using a suitable base such as sodium hydroxide to increase pH and/or a suitable acid such as hydrochloric acid, or may adjusted by other means or with other substances providing for a biocompatible composition.

The composition may comprise one or more other biocompatible compounds or agents. For example, the composition may comprise a biocompatible dye or coloring agent, such as brilliant blue (e.g., Brilliant Blue FCF, also known as FD&C Blue 1) indigo carmine (also known as FD&C Blue 2), indigo carmine lake, FD&C Blue 1 lake, and methylene blue (also known as methylthioninium chloride). Any other suitable types of biocompatible agents may be used, e.g., to adjust the pH and/or tonicity of the composition as appropriate. For example, the composition may comprise one or more stabilizers and/or preservatives. According to some aspects, the composition may comprise an additive such as epinephrine. The composition may include one or more additives that improve visualization of diseased tissue.

A composition comprising carrier 32 may form a gel having a continuous, three-dimensional structure. For example, to prepare the composition, the gelling agent(s) may be combined with at least one pharmaceutically-acceptable salt (and optionally one or more other compounds or agents as discussed above) in an aqueous solution. The resulting mixture may be heated at a temperature ranging from about 70° C. to about 130° C., such as from about 80° C. to about 125° C., from about 90° C. to about 115° C., or from about 95° C. to about 105° C., e.g., a temperature of about 70° C., about 75° C., about 80° C., about 85° C., about 90° C., about 95° C., about 100° C., about 105° C., about 110° C., about 115° C., about 120° C., about 125° C., or about 130° C. In some examples, a minimum temperature from about 70° C. to about 85° C. may be used. In some examples, the mixture may be heated to boiling, e.g., a temperature 100° C.

The composition may be sterilized according to any suitable method, e.g., autoclaving, gamma irradiation, or via electron beam. In at least one embodiment, the composition may be heated at a temperature sufficient for sterilization, e.g., autoclaved at a temperature of about 121° C. The composition may be sterilized according to any suitable method, e.g., autoclaving, gamma irradiation, or via electron beam.

The mixture may be heated for an amount of time sufficient to hydrate the gelling agent and allow for formation of the three-dimensional gel network. With respect to polysaccharides like gellan gum, it is believed that the polysaccharide molecules may undergo a coil to double-helix transition with decreasing temperature, which may lead to gel formation, e.g., depending on the ionic strength and pH of the solution. For example, gellan gum coil molecules may form double helices with a reduction in temperature, and these helices may aggregate to form junction zones, resulting in gelation. In water, at low ionic strength and neutral pH, aggregation of the helices may be impeded by electrostatic repulsion between negatively charged carboxylic groups on the gellan molecules. The addition of a salt and/or the reduction in pH may decrease intermolecular repulsion between the helices, thereby enhancing junction zone formation, and consequently, the gel strength. The addition of salt therefore may facilitate physical cross-linking in an aggregation-like process to form a continuous, three-dimensional gel network. This continuous, three-dimensional network may provide for a solid or quasi-solid gel capable of maintaining its three-dimensional form even when inverted while in an open container.

In some aspects of the present disclosure, the mixture of gelling agent(s) and salt solution may be heated for a time ranging from about 5 minutes to about 90 minutes, from about 10 minutes to about 60 minutes, from about 15 minutes to about 45 minutes, or from about 20 minutes to about 30 minutes, e.g., about 15 minutes, about 20 minutes, about 30 minutes, or about 45 minutes. The mixture may be heated with constant or intermittent stirring, e.g., with a magnetic stirrer or other appropriate mixing equipment. While heated the composition may form a low viscosity fluid. The heat then may be removed and the composition allowed to cool. For example, the composition may be cooled to a temperature 55° C. or about 50° C. As the composition cools, it may increase in viscosity and set into a gel.

In some embodiments, the composition is allowed to cool without stirring or other agitation. In such cases, the composition may form a substantially homogeneous gel, e.g., a continuous solid. Thus, for example, the composition may have a substantially continuous, three-dimensional, solid or quasi-solid gel network, as opposed to an agglomerate of gel particles or a colloid mixture. In some embodiments, the composition may be agitated as it cools, e.g., by constant or intermittent stirring. In such cases, the agitation may at least partially disrupt the structure of the gel, e.g., breaking apart the three-dimensional network to form individual gel particles or gel fragments. Additionally or alternatively, the structure of the gel may be at least partially disrupted after the composition cools, e.g., by stirring, shaking, and/or transferring the composition between containers.

Carrier 32 may include one or more treatment materials or agents such as a thrombogenic or a therapeutic agent, that induces a tissue reaction when placed within a body. When device 10 is placed in a body, carrier 32 may degrade or dissolve and release the treatment agent to its surrounding environment. A body temperature and/or a reaction with a bodily fluid may cause carrier 32 to degrade or dissolve, thereby releasing the agent. Carrier 32 may degrade or dissolve over a period of time such as days, weeks, or months. The rate of degradation or dissolution may vary depending on the composition of carrier 32, the agent used, and/or a geometry of device 10.

The therapeutic agent may be a type which elicits a tissue reaction when exposed to the environment within a body. Exemplary agents or materials may include thrombogenic materials; cytokines; extracellular matrix molecules (e.g., collagen, fibrin, or decellularized animal tissues); matrix metalloproteinase inhibitors; trace metals (e.g., copper); other molecules that may stabilize thrombus formation or inhibit clot lysis (e.g., proteins, including Factor XIII, α2;-antiplasmin, plasminogen activator inhibitor-1 (PAI-1), and the like); and their functional fragments (e.g., the P1 or P2 epitopes of fibrin). Examples of cytokines that may be used alone or in combination with other compounds may include basic fibroblast growth factor (bFGF), platelet derived growth factor (PDGF), vascular endothelial growth factor (VEGF), transforming growth factor beta (TGF-β), and the like.

Bioactive polypeptides may also be synthesized recombinantly for use in carrier 32. Agents may include use of DNA or RNA encoded bioactive molecules. Furthermore, molecules having similar biological activity as wild-type or purified cytokines, extracellular matrix molecules, matrix metalloproteinase inhibitors, thrombus-stabilizing proteins (e.g., recombinantly produced or mutants thereof), and nucleic acid encoding these molecules may also be used. The agent may also include drugs, proteins, cells, genetic modifiers, inflammatory agents, immuno-agonistic agents (e.g. Freunds advuvant or squalene), clot stabilizer, clot activators (e.g. thrombin or Factor XIII), cellular materials (e.g. concentrated blood products, fibroblasts, smooth muscle cells, progenitor cells, genetically engineered cells that secrete a particular bioactive protein), viral vectors, or plasmids. The agent may alternatively be an antibiotic.

The agent may alternatively or additionally include one or more chemotherapy drugs, such as, for example, Cisplatin, Carboplatin, Paclitaxel (Taxol®), Albumin-bound paclitaxel (nab-paclitaxel, Abraxane®), Docetaxel (Taxotere®), Gemcitabine (Gemzar®), Vinorelbine (Navelbine®), Irinotecan (Camptosar®), Etoposide (VP-16®), Vinblastine, and Pemetrexed (Alimta®). The dosage of such chemotherapy and other agents is limited only by internal volume of the enclosing coils, thereby facilitating higher dosages than may be available with other methods, such as methods using microspheres. The elution rate of agents may be controlled not only by the specific composition of the agent but also by the geometry of the enclosing coil and the implant site location chosen.

The amount and concentration of agents that may be included in the composition of carrier 32 may vary depending upon the specific application. It will be understood that any combination of materials, concentration, and/or dosage may be used, depending on the condition, treatment objective, and/or individual patient need.

Carrier 32 may be placed in lumen 30 before, during, or after cooling of carrier 32. Carrier 32 may set in lumen 30. Windings 16 of coil 12 may serve to contain carrier 32. Device 10 may be distributed with carrier 32 pre-infused into lumen 30 of device 10. For example, carrier 32 may be inserted by, for example, injection, into lumen 30 through an open end of coil 12 prior to attachment of a tip 22 or a first portion 26 of delivery device 24. In the alternative, a tip 22 or a first portion 26 of delivery device 24 may be removable in order to facilitate insertion of carrier 32 into lumen 30. In the alternative, an opening 21 may be disposed in a tip 22 or a first portion 26 of delivery device 24, the opening being in fluid communication with lumen 30, to permit injection or other insertion of carrier 32 into lumen 30. Carrier 32 may also be inserted via spaces 34. Device 10 may also be immersed in carrier 32 before, during, or after cooling of carrier 32. Carrier 32 may be set in lumen 30, carrier 32 may coat an exterior of device 10, or carrier 32 may both be set in lumen 30 and coat an exterior of device 10. Carrier 32 may set up or cure in lumen 30, set up or cure as a coating on exterior of device 10, or both set up or cure in lumen 30 and as a coating on an exterior of device 10. In the alternative, carrier 32 may be infused into lumen 30 of device 10 prior to a surgical procedure for implanting device 10 via, for example, the approaches described above. Such infusion may occur immediately before such surgical procedure or some time in advance of such surgical procedure.

In addition to the composition of a gelling agent, at least one salt, and water, carrier 32 may also include an axially-oriented element 33, which may be located within lumen 30 and may be secured to first and second end portions 18 and 20 or to one or more tips 22. The securing may be accomplished by an anchor or a suitable adhesive, such as ultraviolet-curable adhesives, silicones, cyanoacrylates, or epoxies. Alternatively, the axially-oriented element can be secured to the coil 12 by chemical bonding between reactive groups on the axially-oriented element and the coil 12, solvent bonding, fusing both materials so that they melt together, or temporarily melting the surface of the coil 12 to embed part of the axially-oriented element.

An axially-oriented element may function as a stretch-resistant member, which prevents the first end portion 18 from being pulled too far from the second end portion 20. The axially-oriented element can also be pre-stretched before it is secured to the ends of the coil 12, to thereby provide some degree of compression within the coil 12. In the alternative, the axially-oriented element can be secured to the coil 12 at one of the end portions 18 and 20 or at one or more points along a length of the coil 12 by a suitable adhesive or by wrapping around one or more windings 16 of the coil 12. In another alternative, the axially-oriented element is not secured to the coil 12, but is simply disposed within the lumen 30 or is coupled to the coil 12 by a surface friction, in which case, the surface of the axially-oriented element may be textured to improve the coupling force between the axially-oriented element and the coil 12.

The axially-oriented element may have a cross-sectional dimension such that the overall flexibility of the device is not significantly impacted. In one embodiment, the cross-sectional dimension of the axially-oriented element may be approximately 0.002 inch less than the internal diameter of the lumen 30. However, any diameter smaller than the lumen diameter may also be used. If the axially-oriented element is also used as a stretch-resistant member, the axially-oriented element may have a minimum cross-sectional dimension such that the axially-oriented element may have enough strength to provide some degree of tensile resistance to a stretching of the coil 12.

Carrier 32 may at least partially fill the volume of lumen 30 between an outer surface of the axially-oriented element and an interior surface of coil 12. For example, carrier 32 may form a cylindrical sheath surrounding the axially-oriented element. It is also contemplated that the axially-oriented element also be configured to elute a treatment agent, which may be the same or different from the treatment agent in carrier 32.

Device 10 may also include another carrier for use in combination with carrier 32. For example, a surface of device 10, for example, a surface of coil 12 or tip 22, may be infused with, coated with, or wrapped with a material which is biodegradable, dissolvable, or absorbable when device 10 is implanted in a body lumen. A material coating a surface of device 10 may be similar to carrier 32 or may differ from carrier 32. A material coating a surface of device 10 may include a treatment agent, which may be the same as or similar to the treatment agent carried by carrier 32. In the alternative, a material coating a surface of device 10 may include a different treatment agent. A treatment agent may be chosen from any of the treatment agents discussed above. A material coating a surface of device 10 may treat a condition in conjunction with a carrier 32 and/or a physical characteristic of device 10.

Device 10 may thus have multiple operative pathways. A device 10 may, for example, occlude a lumen. For example, a device 10 implanted in a blood vessel may reduce blood flow to a target location. A device 10 may also elute a therapeutic agent via carrier 32 or via a coating. Device 10 may also act as a fiducial marker in order to identify where a therapeutic agent has been applied.

Spaces 34 may exist between windings 16. Where coil 12 is close-coiled or relatively close-coiled, carrier 32 may be relatively contained within lumen 30 because spaces 34 are relatively small. In other words, carrier 32 may be kept on the interior of coil 12 and may not be visible (e.g., with the naked eye) from the exterior of coil 12 through spaces 34. In the alternative, carrier 32 may be visible through relative larger spaces 34 between windings 16 of coil 12. Where carrier 32 is not visible through spaces 34, carrier 32 may nevertheless be able to elute (e.g., leak or otherwise pass) through spaces 34, or bodily fluids may be able to pass through spaces 34 or other portions of device 10 in order to access carrier 32 and facilitate elution. Where carrier 32 is visible through spaces 34, carrier 32 may elute out of lumen 30 more easily. The size of spaces 34 between windings 16 of coil 12 may vary along the length of coil 12. For example, as shown with regard to device 10, coil 12 is relatively more close-coiled in the linear portion 36 of device 10 and is relatively more open-coiled in the curved portions 38 of device 10. Spaces 34 thus may be smaller in the linear portion 36. Carrier 32 and/or bodily fluids may therefore more easily pass through spaces 34 in curved portion 38 of spaces 34 in linear portion 36 of device 10.

An agent in carrier 32 may be more quickly dispersed or otherwise administered in portions of device 10 where spaces 34 are larger. For example, carrier 32 may degrade and/or a corresponding agent may be administered more quickly in curved portions 38 than in linear portion 36. The differences in elution rates between portions of device 10 may be enhanced, permitted, mitigated, or eliminated, depending on need, by modifying one or more characteristics of coil 12 and/or carrier 32. Examples of characteristics that may be modified have been described in the preceding paragraphs. Other examples of characteristics that may be modified are described in the paragraphs below.

FIG. 2 depicts an alternative device 50 for delivery of one or more treatment agents. Device 50 may have any of the features described above with regard to device 10 as depicted in FIG. 1. The same is true for the other devices depicted herein. Device 50 may include a coil 52. Coil 52 may be the same as or similar to coil 12 as described with regard to FIG. 1 in one or more respects of composition and construction; and along the same lines, coil 12 may be the same or similar to coil 52 in composition and construction. Device 50 may have two tips 22 at a first end 54 and a second end 56 of device 50. As with device 10 as depicted with regard to FIG. 1, device 50 may be of any suitable shape. A lumen 58 may have features that are the same as or similar to lumen 30 as described with regard to FIG. 1. A carrier 32 as described with regard to FIG. 1 may be disposed in lumen 58.

Coil 52 may be close-coiled or open-coiled. If coil 52 is close-coiled, windings 60 may abut one another. If coil 52 is open-coiled, the spaces 62 between windings 60 may be relatively larger. The windings 60 of coil 52 may be more open or more closed at different locations along the length of coil 52 between first end 54 and second end 56. That is, spaces 62 between windings 60 may be smaller or larger in different portions of coil 52. For example, as described above with regard to coil 12, spaces 62 may be larger in curved portion 64 than in linear portion 66 of device 50. Spaces 62 may otherwise vary in any suitable manner. For example, portion 68 of coil 52 may be relatively more close-coiled. That is, spaces 62 may be relatively smaller, or windings 60 may abut one another. Portion 70 of coil may be relatively more open-coiled. That is, spaces 62 may be relatively larger, exposing more of carrier 32. Carrier 32 and/or bodily fluids may therefore more easily pass through spaces 62 in portion 70 than in portion 68. An agent in carrier 32 may be more quickly dispersed or otherwise administered in portion 70 than in portion 68.

Coil 52 may alternate between sections which are relatively more closed coiled and sections which are relatively more open-coiled. For example, windings 60 may be arranged so that spaces 62 between windings 60 vary according to any pattern or randomly. For example, winding 72 may be separated from winding 74 by a relatively larger space 76. Winding 74 may be separated from winding 78 by a relatively smaller space 80. Winding 78 may be separated from winding 82 by a relatively larger space 84. There may be a pattern of relatively smaller and larger spaces 62. Alternatively, there may be no pattern underlying the distribution of relatively smaller and larger spaces 62. Numerous consecutive windings may be separated by a relatively smaller space 62 or by a relatively larger space 62. For example, a number of relatively smaller spaces 62 may be followed by a number of relatively larger spaces 62. The size of spaces 62 may vary in both or either of curved portion 64 and/or linear portion 66. The sizes of spaces 62 between windings 60 may be chosen to release a treatment agent carried by carrier 32 at a desired rate. A treatment agent may be released at a constant or a variable rate by varying the above-described characteristics.

Additionally or alternatively, device 50 may have other characteristics which cause variations along device 50. For example, coil 52 may include a wire which has a varying diameter. For example, in one region of device 50, a wire forming coil 52 may have a relatively narrow diameter. In another region of device 50, the wire may have a relatively wider diameter. For example, a diameter of a wire may vary by any appropriate multiple. For example, a diameter in a second region of device 50 may be 1.1 times to 5 times as large as a diameter in a first region of device 50. Multiple different diameters of a wire may be used to create numerous regions. Use of different wire diameters may result in, for example, different spaces between windings. For example winding 72 may be separated from winding 74 by a relatively larger space 76 because a particular diameter of wire is used. Winding 74 may be separated from winding 78 by a relatively smaller space 80 as a result of a diameter of a wire in that region. It is also contemplated that the sue of different wire diameters in different regions may result in lumen 58 having different widths or diameters in those regions. Additionally or alternatively, different diameters of a wire may result in the same width of spaces 62 but may otherwise affect elution rate of carrier 32.

Additionally or alternatively, a device 50 may have different widths along a length of device 50. For example, lumen 58 may have a different diameter or width along a length of device 50. For example, a diameter or width of lumen 58 in portion 70 may be different from a diameter or width of lumen 58 in portion 68. Any number of different portions with varying widths may be used. Portions of varying widths may be located anywhere along a length of a device 50 and may be of any length. For example, portions of varying widths may be the same length or of different lengths.

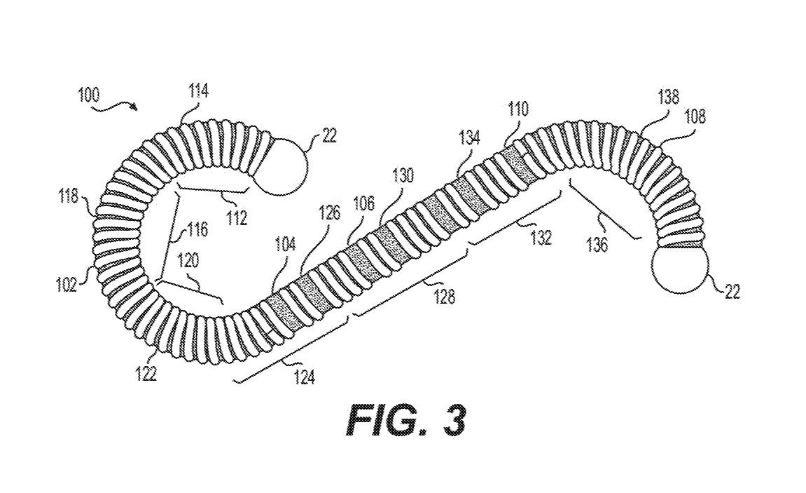

FIG. 3 depicts an alternative device 100 for delivery of one or more treatment agents. Device 100 may have any of the features described above with regard to device 10 as depicted in FIG. 1 or device 50 as depicted in FIG. 2, and/or vice-versa. Device 100 may include a coil 102 defining a lumen 104. Coil 102 may be the same as or similar to coils 12 and 52 as described with regard to FIGS. 1-2, and/or vice-versa. Lumen 104 may be the same as or similar to lumen 30 or lumen 58 as described with regard to FIGS. 1-2, and/or vice-versa. Coil 102 may be relatively more open-coiled or relatively more close-coiled, as described with regard to coils 12 and 52. The spaces 106 between windings 108 may be arranged as described with regard to FIGS. 1-2, and/or vice-versa.

Carrier 110 may be disposed within lumen 104. Carrier 110 may be the same as or similar to carrier 32 and may carry one or more treatment agents, and/or vice-versa. Portions of carrier 110 located at different regions of coil 102 may have different properties. The below discussion will reference regions as indicated on FIG. 3. These regions are exemplary only. Coil 102 may be subdivided by any suitable method. For example, in region 112, a carrier 114 may be disposed in a portion of lumen 104. In region 116, a carrier 118 may be disposed within a portion of lumen 104. Carrier 118 may be the same as or similar to carrier 114, or may be different from carrier 114 in one or more ways with respect to their composition. Carrier 118 and carrier 114 may be infused with the same or similar treatment agent. In the alternative, different treatment agents may be carried by carrier 114 and carrier 118. Similarly, region 120, region 124, region 128, region 132, and/or region 136 may include carrier 122, carrier 126, carrier 130, carrier 134, and/or carrier 138, respectively. Any of carrier 114, carrier 118, carrier 122, carrier 126, carrier 130, carrier 134, and/or carrier 138 may be the same as, similar from, or different to, any of the other carriers.

Carriers 114, 118, 122, 126, 130, 134, and/or 138 may be infused into lumen 104 according to any of the methods described above with regard to FIG. 1. Carriers 114, 118, 122, 126, 130, 134, and/or 138 may be introduced concurrently or sequentially. Carriers 114, 118, 122, 126, 130, 134, and/or 138 may be infused into lumen 104 over any time period. For example, a carrier may be permitted time to set before introducing another carrier. Carriers 114, 118, 122, 126, 130, 134, and/or 138 may be of varying colors in order to aid in distinguishing between them during infusion and/or during implantation of device 100 in a body lumen. In addition or in the alternative, device 100 may have different colored portions in order to distinguish between regions of coil 102. For example, one or more windings 108 may be of different colors. For example, a particular region may be denoted by one or more colored windings 108. In another alternative, one or more windings 108 of a region may include a radiopaque material. Such radiopaque material may be used to distinguish between regions of coil 102.

There may be any number of regions of coil 102. Regions may be of any size and may be the same size or different sizes. There can be more than one region in a particular cross-section of lumen 104. The regions of coil 102 and their corresponding carriers may be chosen for anatomical or other reasons. For example, it may be desirable to deliver an agent to a location of a body where a region will be located after a device 100 is implanted. Carriers located in different regions may release one or more treatment agents at different rates. It may be desirable to keep a treatment agent apart from another treatment agent until the two agents are released into the body. Therefore, the treatment agents may be in different carriers. The one or more carriers and treatment agents may have any of the characteristics as described with regard to carrier 32 as described with regard to FIGS. 1-2, and/or vice-versa. The one or more carriers may include the same or different components in the same or different proportions. One or both of a carrier material and a treatment agent may vary. In certain regions, a carrier may lack an active agent. Carrier 32 may interact with an external environment to aid elution and determine release rate. For instance, composition of carrier 32 may be such that exposure to a particular pH once device 10 is implanted will accelerate degradation of carrier 32 and thereby accelerate treatment agent release rate. By way of further example, geometry of device 10 may be such that an external flow rate over device 10 is utilized to accelerate degradation of carrier 32 and also to disperse treatment agent along a fluid path. As a further example, composition of carrier 32 may be such that it is hypertonic and thus utilizes relative osmotic pressure to accelerate absorption of a treatment agent at an implanted location of device 10.

As described above with regard to FIG. 1, one or more devices 10, 50, and/or 100 may include a coating in one or more regions of device 10, 50, and/or 100. One coating may be applied to all regions of devices 10, 50, and/or 100. In the alternative, a coating may be used in one or more regions of devices 10, 50, and/or 100, while other regions of devices 10, 50, and/or 100 may lack a coating. In yet another alternative, different coatings may be used in different regions of devices 10, 50, and/or 100. Any combination of different coatings or lack of coatings may be used on a device 10, 50, and/or 100. Coatings used on a device 10, 50, and/or 100 may have a variety of functions. For example, a coating may elute a therapeutic agent, while delaying elution of therapeutic agents from carrier 32, to implement a two-stage treatment process wherein a treatment agent in a coating has an initial effect on a target location and a therapeutic agent in carrier 32 has a follow-on effect on the target location (or vice versa). Carriers having different functions may be used on the same device 10, 50 and/or 100, and a coating may have more than one function. In the alternative, no coating may be used on a device 10, 50, and/or 100.

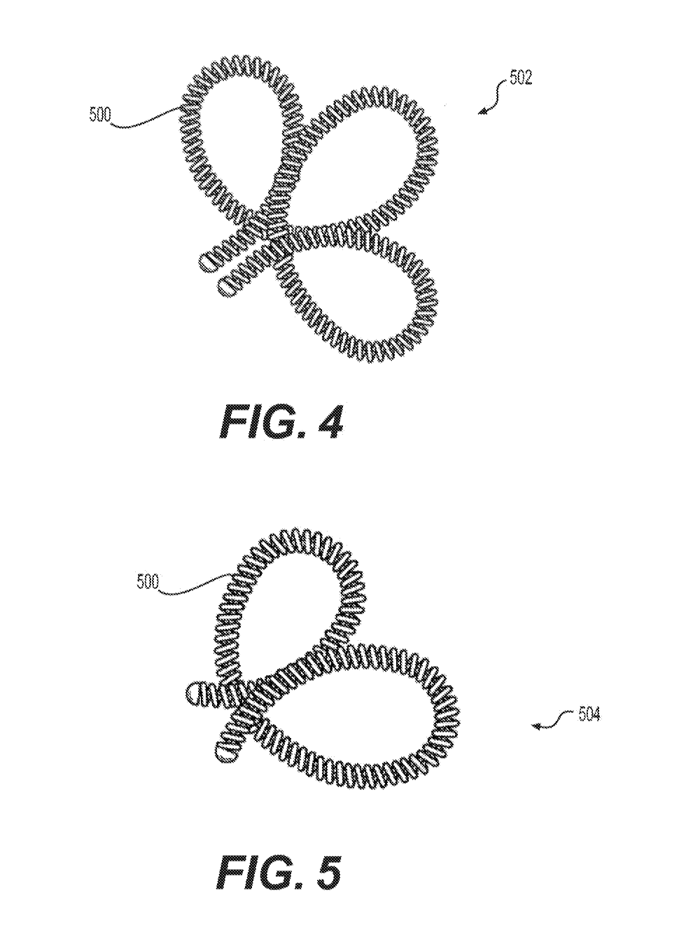

Device 10 (and the other devices discussed herein) may be formed into any suitable shape. For example, as shown in FIG. 1, device 10 may be formed into a curved shape such as an “S” shape. In other words, a central axis of lumen 30 may be shaped like an “S.” In other words, the secondary shape of device 10 may be an “S” shape. Other exemplary shapes are discussed below, with regard to FIGS. 4-10.

FIGS. 4-10 illustrate various devices that include a coil 500 having a primary shape and a secondary shape. FIGS. 4-10 are exemplary of secondary shapes that may be used with the devices described above. These shapes are simply indicative of the various secondary shapes that may be used, and other shapes may be used as well. The devices illustrated in FIGS. 4-10 are each provided with a carrier, which may include a treatment agent, as discussed above in the description for FIGS. 1-3.

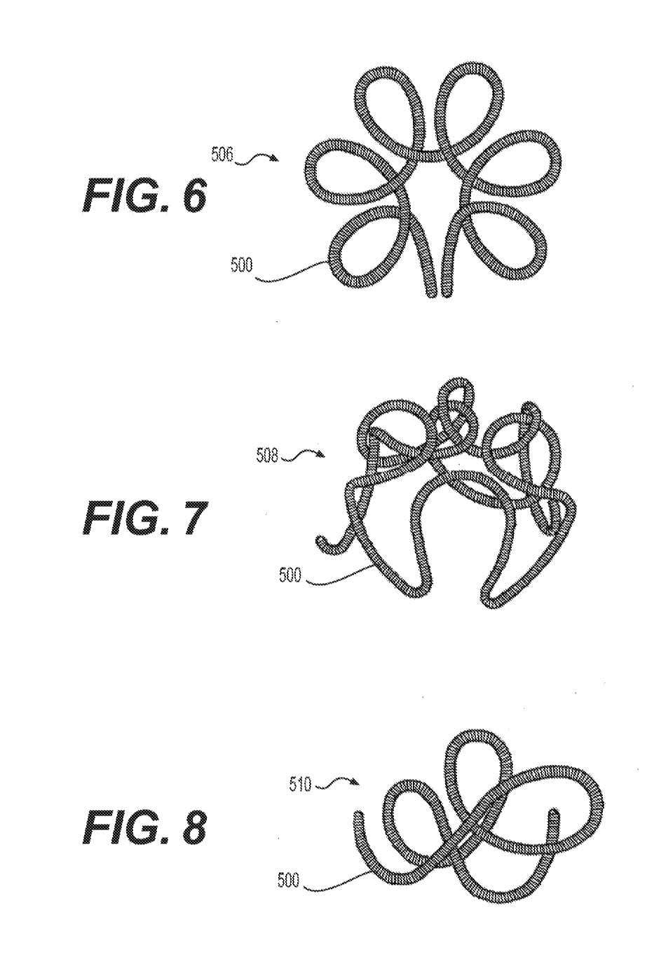

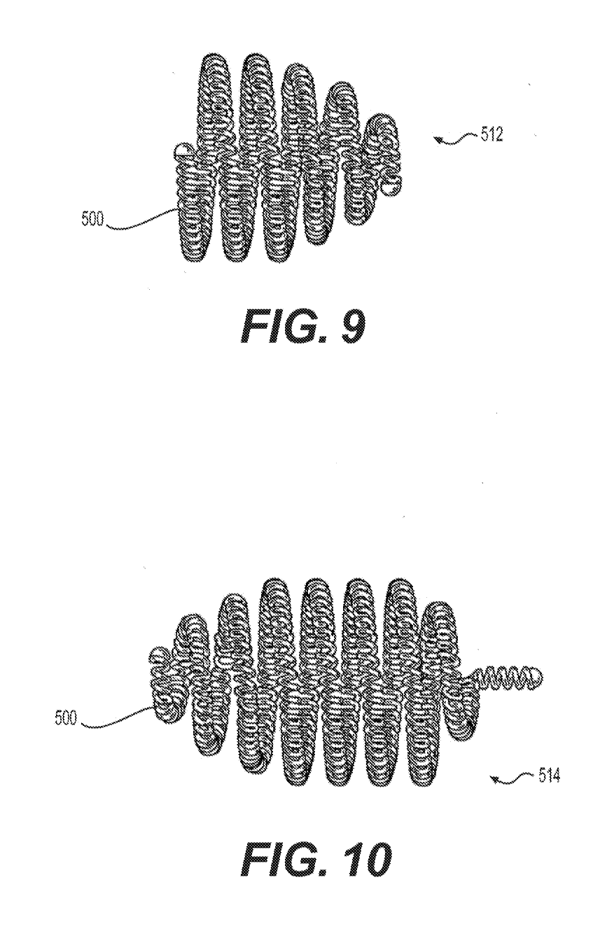

FIG. 4 depicts a device 502 having a secondary shape of a clover leaf. FIG. 5 depicts a device 504 having a secondary shape of a twisted figure-eight. FIG. 6 depicts a device 506 having a flower-shaped secondary shape. FIG. 7 depicts a device 508 having a round secondary shape. A contemplated version of device 508 may have a substantially spherical secondary shape. FIG. 8 illustrates a device 510 having a random or irregular secondary shape. FIG. 9 illustrates a device 512 having a secondary shape of a cone or vortex. FIG. 10 illustrates a device 514 having a secondary shape of an ovoid. It should be noted that the devices described in FIGS. 1-10 may also have other secondary shapes, and that it should not be limited to the examples illustrated previously. For example, the device may be selectively sized and/or shaped to fill a particular body lumen, treatment location, or body cavity.

To make a secondary shaped device as shown in any of the figures described herein, a coil (e.g., the coil 500) having a primary shape that is substantially rectilinear or curvilinear may be wrapped around a mandrel or other shaping element to form a secondary shape. The coil 500 may be heat treated to shape the coil 500 into the secondary shape.

While principles of the present disclosure are described herein with reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device comprising:
a helical coil having a plurality of turns, wherein the turns of the coil define a lumen; and
a composition comprising:
gellan gum,
at least one salt,
water, and
a treatment agent;
wherein the composition is disposed in the lumen.

2. The medical device of claim 1, wherein the treatment agent is a thrombogenic agent or a therapeutic agent.

3. The medical device of claim 1, wherein the helical coil includes at least a first region and at least a second region, and wherein a distance between turns in the first region differs from a distance between turns in the second region.

4. The medical device of claim 3, wherein the composition is disposed in the first region and the second region, wherein the medical device is operative to elute at least a portion of the composition disposed in the first region at a first rate when the device is delivered to a location within a body, wherein the medical device is operative to elute at least a portion of the composition disposed in the second region at a second rate when the device is delivered to the location within the body, and wherein the first rate differs from the second rate.

5. The medical device of claim 3, wherein the distance between turns in the first region is at least approximately 1.5 times as large as the distance between turns in the second region.

6. The medical device of claim 3, wherein the distance between turns in the first region is at least approximately 2.0 times as large as the distance between turns in the second region.

7. The medical device of claim 3, wherein the helical coil includes a third region, and wherein a distance between turns in the third region differs from the distance between turns in the first region and the distance between turns in the second region.

8. The medical device of claim 1, wherein the composition is a first composition and the treatment agent is a first treatment agent, and the medical device further comprises:
a second composition disposed in the lumen,
wherein the first composition differs from the second composition.

9. The medical device of claim 8, wherein the first composition has a first elution rate and the second composition has a second elution rate, and wherein the first elution rate differs from the second elution rate.

10. The medical device of claim 8, wherein the second composition includes a second treatment agent, and the first treatment agent differs from the second treatment agent.

11. The medical device of claim 1, wherein the composition comprises 0.01% to 2.0% gellan gum by weight with respect to a total weight of the composition.

12. A medical device comprising:
an outer region defining a lumen, the outer region terminating at a first free end and at a second free end, the first and second free ends being on opposite ends of the outer region; and
a composition disposed in the lumen comprising:
0.01% to 2.0% gellan gum by weight with respect to a total weight of the composition,
at least one salt,
water, and
a treatment agent
wherein the device is operative to elute the treatment agent through one or more gaps in the outer region.

13. The medical device of claim 12, wherein the outer region includes a first gap and a second gap, wherein the first gap is larger than the second gap.

14. The medical device of claim 12, wherein at least one of the first free end and the second free end include a tip.

15. A method of preparing a medical device, the method comprising:
introducing a composition into the medical device,
wherein the medical device includes a helical coil having a plurality of turns, and wherein the turns of the coil define a lumen for receiving the composition, and
wherein the composition includes:
gellan gum,
at least one salt,
water, and
a treatment agent.

16. The method of claim 15, wherein the introducing step occurs immediately prior to a procedure for implanting the medical device in a body.

17. The method of claim 15, wherein the helical coil includes at least a first region and at least a second region, and wherein a distance between turns in the first region differs from a distance between turns in the second region.

18. The method of claim 17, wherein the introducing step includes infusing the composition in the first region and the second region, wherein the medical device is operative to elute at least a portion of the composition disposed in the first region at a first rate when the device is delivered to a location within a body, wherein the medical device is operative to elute at least a portion of the composition disposed in the second region at a second rate when the device is delivered to the location within the body, and wherein the first rate differs from the second rate.

19. The method of claim 15, wherein the composition is a first composition and the treatment agent is a first treatment agent, and the method further comprises introducing a second composition into the lumen of the medical device, wherein the first composition differs from the second composition.

20. The method of claim 19, wherein the first composition has a first elution rate and the second composition has a second elution rate, and wherein the first elution rate differs from the second elution rate.
